(19) United States
(12) Patent Application Publication
(10) Pub. No.: US 2025/0268673 A1
(43) Pub. Date: Aug. 28, 2025

(54) ROBOTIC SURGICAL INSTRUMENTS

(71) Applicant: Covidien LP, Mansfield, MA (US)

(72) Inventors: Hans Christian Pflaumer, Apex, NC (US); Jack Alexander Hornsby, Tempsford (GB); Matthew M. Marinovich, London (GB); Jozsef Horvath, Wake Forest, NC (US); Liam M. Connelly, Meldreth (GB)

(21) Appl. No.: 18/857,442

(22) PCT Filed: May 10, 2023
(86) PCT No.: PCT/CA2023/050636

§ 371 Date: Oct. 16, 2024

Related U.S. Application Data
(60) Provisional application No. 63/342,272, filed on May 16, 2022.

(51) Int. Cl. A61B 34/37; A61B 34/00

(52) U.S. Cl. CPC A61B 34/37; A61B 34/71

(57) ABSTRACT

A robotic surgical instrument includes an instrument cassette assembly, an elongated shaft assembly extending from the instrument cassette assembly, and an end effector supported on the elongated shaft assembly. The elongated shaft assembly defines a longitudinal axis. The instrument cassette assembly includes a cable drive assembly, a grip mechanism, a manual release mechanism, and a wrist mechanism. The cable drive assembly includes a drive dog assembly having a drive dog pinion, a first rack assembly, and a second rack assembly. The first and second rack assemblies translate relative to one another in response to rotation of the drive dog pinion to cause the end effector to move relative to the longitudinal axis. The grip and wrist mechanisms are coupled to the end effector and the manual release mechanism is coupled to the grip mechanism.

### TECHNICAL FIELD

This disclosure relates to robotic systems and, more particularly, to robotic surgical instruments.

### BACKGROUND

Surgical instruments used in laparoscopic and/or robotic surgery generally have a proximal housing and an elongated shaft extending distally from the proximal housing to an end effector. The proximal housing supports actuating mechanisms that may be used to actuate the end effector for performing a surgical task within a body cavity of a patient. Such instruments may be used in applications where there is an area of limited access for an operator. The end effector may be inserted into the area of limited access and the operator may remotely and/or robotically manipulate the instrument via the actuating mechanisms.

### SUMMARY

In accordance with an aspect of this disclosure, a robotic surgical system includes a surgical instrument for a robotic surgical system. The surgical instrument includes an end effector, and elongated shaft assembly, and an instrument cassette assembly. The elongated shaft assembly defines a longitudinal axis and has a distal end portion and a proximal end portion. The distal end portion includes a dexterous assembly supporting the end effector. The dexterous assembly is movable relative to the longitudinal axis to move the end effector in different planes. The instrument cassette assembly is supported on the proximal end portion of the elongated shaft assembly. The instrument cassette assembly includes a cable drive assembly including a plurality of drive dog assemblies. Each drive dog assembly of the plurality of drive dog assemblies includes a drive dog pinion, a first rack assembly, and a second rack assembly. The first rack assembly includes a first cable, and the second rack assembly includes a second cable. The first and second rack assemblies are positioned to translate relative to one another in response to rotation of the drive dog pinion to cause the first and second cables to move the end effector relative to the longitudinal axis.

In aspects, the first rack assembly may include a first ferrule that secures the first cable to a first rack of the first rack assembly. The first rack assembly may include a clip that is removably coupled to the first rack to secure the first ferrule to the first rack.

In aspects, the instrument cassette assembly may include a transition block that directs the first and second cables into the elongated shaft assembly. The transition block may include a plurality of shells secured together to define cable channels through the transition block, wherein the cable channels define a longitudinal radial pattern that is configured to direct the first and second cables into a shaft hub assembly supported on the proximal end portion of the elongated shaft assembly.

In aspects, the instrument cassette assembly may include a wrist mechanism. The wrist mechanism may include a double-sided rack, a wrist drive dog pinion, and a wrist gear. The wrist drive dog pinon may be rotatable to translate the double-sided rack. The wrist gear may be rotatable in response to translation of the double-sided rack. The wrist mechanism may further include a compound shaft assembly coupled to the wrist gear. The compound shaft assembly may extend through the elongated shaft assembly and may include one or more flexible sections and one or more rigid sections. The wrist gear may be disposed at an angle relative to the wrist drive dog pinon.

In aspects, the instrument cassette assembly may include a grip mechanism that is actuatable to move jaw members of the end effector relative to another, and a manual release mechanism that is coupled to the grip mechanism.

According to one aspect, this disclosure is directed to an instrument cassette assembly of a robotic surgical instrument. The instrument cassette assembly includes a housing assembly, a cable drive assembly, a grip mechanism, and a release mechanism. The housing assembly supports a mounting plate. The cable drive assembly is supported in the housing assembly and includes a drive dog pinion, a first rack assembly, and a second rack assembly. The first and second rack assemblies are enmeshed with the drive dog pinion and positioned to translate relative to one another in response to rotation of the drive dog pinion. The grip mechanism is supported on the mounting plate and the release mechanism is coupled to the grip mechanism.

In aspects, the housing assembly may include a cover that is selectively removable from the housing assembly to expose the release mechanism. The release mechanism may include a lock sled, a carriage, a dial, and a spring. The lock sled may be slidably movable relative to the carriage to enable the spring to urge the carriage and the dial from a first position to a second position. The dial may be rotatable relative to the carriage in the second position to operate the grip mechanism.

In aspects, the grip mechanism may include a drive dog and a crank operatively associated with the drive dog. The crank may be configured to receive a grip cable therethrough.

In aspects, the instrument cassette assembly may include a high voltage circuit supported within the housing assembly.

In aspects, the instrument cassette assembly may include a latch assembly supported by the housing assembly, the latch assembly being actuatable to disconnect a sterile adapter assembly from the housing assembly. The latch assembly may include a latch spring coupled to a pair of latches. The pair of latches may be configured to secure the housing assembly to the sterile adapter assembly.

In aspects, the instrument cassette assembly may include the sterile adapter assembly.

According to another aspect, this disclosure is directed to a robotic surgical system. The robotic surgical system includes an instrument drive assembly, a sterile adapter assembly coupled to the instrument drive assembly, and a robotic surgical instrument coupled to the sterile adapter assembly and actuatable by the instrument drive assembly. The robotic surgical instrument includes an instrument cassette assembly, an elongated shaft assembly extending from the instrument cassette assembly, and an end effector supported on a distal end portion of the elongated shaft assembly. The elongated shaft assembly defines a longitudinal axis. The instrument cassette assembly includes a cable drive assembly, a grip mechanism, a manual release mechanism, and a wrist mechanism. The cable drive assembly includes a plurality of drive dog assemblies, each drive dog assembly of the plurality of drive dog assemblies including a drive dog pinion, a first rack assembly, and a second rack assembly. The first and second rack assemblies are positioned to translate relative to one another in response to rotation of the drive dog pinion to cause the end effector to move relative to the longitudinal axis. The grip mechanism is operatively coupled to the end effector and actuatable to cause jaw members of the end effector to move between an open position, a closed position, and an overclosed position. The manual release mechanism is operatively coupled to the grip mechanism to enable the jaw members to be manually moved relative to one another. The wrist mechanism is operatively coupled to the end effector and actuatable to cause the end effector to rotate.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate aspects of this disclosure and, together with a general description of this disclosure given above, and the detailed description given below, explain the principles of this disclosure, wherein:

FIG. 1 is a perspective view of a robotic surgical system being used for a surgical procedure on a patient in accordance with the principles of this disclosure;

FIGS. 2-4 are progressive views illustrating surgical instruments of the robotic surgical system of FIG. 1 being manipulated within a body cavity of the patient;

FIG. 5 is a perspective view of one robotic surgical instrument of the robotic surgical system of FIG. 1;

FIG. 6 is an enlarged, rear, perspective view of a proximal portion of the robotic surgical instrument of FIG. 5;

FIG. 7 is a perspective view, with parts separated, of an instrument cassette assembly of the robotic surgical instrument of FIG. 5, the instrument cassette assembly shown coupled to a shaft hub assembly of an elongated shaft assembly of the robotic surgical instrument;

FIGS. 8 and 9 are perspective, front views of the instrument cassette assembly of FIG. 7 with portions thereof removed for clarity;

FIG. 10 is an enlarged, perspective, rear view of FIG. 8;

FIG. 11 is an enlarged, perspective view of a cable drive assembly of the instrument cassette assembly of FIG. 7;

FIG. 12 is a perspective view, with parts separated, of FIG. 11;

FIG. 13 is an enlarged, perspective view of a rack assembly of the cable drive assembly of FIG. 11;

FIG. 14 is an enlarged, perspective view of a transition block assembly of the instrument cassette assembly of FIG. 7;

FIG. 15 is an enlarged, perspective, front view of a portion of a transition block of the transition block assembly of FIG. 14 and illustrating an arrangement of distal cable apertures defined by the transition block;

FIG. 16 is a schematic view illustrating a cable routing arrangement of cables of the transition block assembly through the distal cable apertures shown in FIG. 15;

FIG. 17 is a perspective view illustrating the transition block assembly of FIG. 14 coupled to the shaft hub assembly of FIG. 7;

FIG. 18 is a perspective view, with parts separated, of FIG. 17;

FIG. 19-25 are progressive views illustrating movement of drive dogs of a drive assembly of robotic surgical instrument of FIG. 5 causing corresponding movement of an end effector of the robotic surgical instrument;

FIG. 26 is a schematic view illustrating a compounded shaft assembly within the robotic surgical instrument of FIG. 5;

FIG. 27 is an enlarged, perspective view of a wrist mechanism of the robotic surgical instrument of FIG. 5;

FIG. 28 is a side view of FIG. 27;

FIG. 29 is a perspective view of the instrument cassette assembly of FIG. 7 with a cover thereof shown separated and with a grip and wrist hub assembly of the instrument cassette assembly shown in broken lines for clarity;

FIGS. 30-32 are an enlarged, perspective views of the grip and wrist hub assembly of FIG. 29;

FIGS. 33-39 are progressive views illustrating operation of a manual release mechanism of the grip and wrist hub assembly of FIG. 29;

FIG. 40 is a perspective view of portions of the instrument cassette assembly of FIG. 7 and illustrating a high voltage circuit thereof;

FIG. 41 is a schematic view of the high voltage circuit of FIG. 40;

FIG. 42 is a perspective view illustrating the robotic surgical instrument of FIG. 5 being attached to a sterile adapter assembly;

FIG. 43 is rear, perspective view of a proximal portion of the instrument cassette assembly of FIG. 7 and illustrating a latch assembly thereof;

FIG. 44 is perspective view of the latch assembly of FIG. 43; and

FIG. 45 is an enlarged perspective view of the indicated area of detail shown in FIG. 42.

### DETAILED DESCRIPTION

Aspects of this disclosure are described in detail with reference to the drawings, in which like reference numerals designate identical or corresponding elements in each of the several views. As used herein, the term “distal” refers to that portion of structure farther from the user (e.g., clinician), while the term “proximal” refers to that portion of structure, closer to the user. As used herein, the term “clinician” refers to a doctor, nurse, or other care provider and may include support personnel and/or equipment operators. As used herein, the term “cable” refers to one or more wires or fibers that may include metallic or nonmetallic materials, that may have one or more protective casings or insulation (e.g., polymeric material such as rubber or plastic) thereon, and/or that may be twisted together. In some aspects, cables may include one or more nitinol wires.

In the following description, well-known functions or constructions are not described in detail to avoid obscuring the present disclosure in unnecessary detail.

Robotic surgical systems have been used in minimally invasive medical procedures and can include robotic arm assemblies. Such procedures may be referred to as what is commonly referred to as “Telesurgery.” Some robotic arm assemblies include one or more robot arms to which surgical instruments can be coupled. Such surgical instruments include, for example, endoscopes, electrosurgical forceps, cutting instruments, staplers, graspers, electrocautery devices, or any other endoscopic or open surgical devices. Prior to or during use of the robotic surgical system, various surgical instruments can be selected and connected to the robot arms for selectively actuating end effectors of the connected surgical instruments.

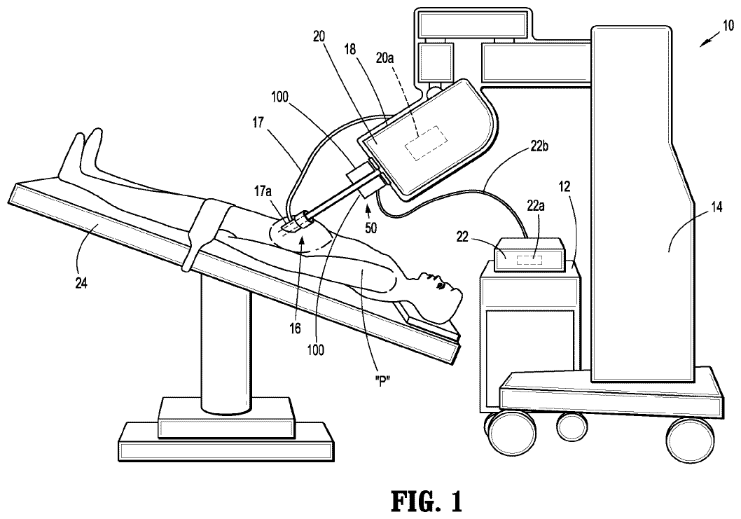

With reference to FIGS. 1-4, a robotic surgical system is shown generally at 10. Robotic surgical system 10 employs various robotic elements to assist the clinician and allow remote operation (or partial remote operation) of surgical instruments 100 of surgical instrument systems 50 of robotic surgical system 10. Various controllers, circuitry, robotic arms, gears, cams, pulleys, electric and mechanical motors, etc. may be employed for this purpose and may be designed with surgical system 10 to assist the clinician during an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

Robotic surgical system 10 includes a workstation 12 and an instrument cart 14. The instrument cart 14 includes one or more surgical instrument systems 50 mounted on a moveable drive unit 18 that houses an instrument drive assembly 20 for manipulating the surgical instrument system 50 and/or independent surgical instruments 100 thereof with the assistance of, for example one or more computing devices or controllers. The surgical instruments 100 can include, for example, graspers or forceps 26, which may be electrosurgical, an endoscope 28, and/or any other suitable instrument that can be driven by one or more associated tool drives (not shown) of instrument drive assembly 20. For example, besides graspers 26 and endoscope 28, the one or more surgical instruments 100 can include dexterous tools, such as needle drivers, staplers, dissectors, cutters, hooks, scissors, coagulators, irrigators, suction devices, etc., or combinations thereof, that are used for performing a surgical procedure.

Surgical instrument system 50 includes an insertion tube 16 defining a plurality of separate conduits, channels or lumens 16a therethrough that are configured to receive, for instance, surgical instruments 100 for accessing a body cavity “BC” of a patient “P.” In other aspects, the insertion tube 16 may define a single conduit, channel or lumen therethrough that is configured to receive, for instance, the surgical instruments 100 for accessing a body cavity “BC” of a patient “P.” In particular, the insertion tube 16 can be inserted through an incision “I” and/or access device 17a, 17b (e.g., a surgical portal, which may include or more seals to facilitate sealed insertion through tissue “T” of the patient “P”) and into the body cavity “BC” of the patient “P”. With insertion tube 16 positioned in the patient “P,” the surgical instruments 100 can be advanced through insertion tube 16 into the body cavity “BC” of the patient “P.” In some aspects, insertion tube 16 may be in the form of an access device, such as access device 17a, and movable drive unit 18 may include a support arm 19 that is selectively attachable to access device 17a so that surgical instruments 100 can be advanced through access device 17a when access device 17a is coupled to a distal end portion of support arm 19.

Further, the workstation 12 includes an input device 22 for use by a clinician for controlling the surgical instrument system 50 and surgical instruments 100 thereof via the instrument drive assembly 20 to perform surgical operations on the patient “P” while the patient “P” is supported on a surgical table 24, for example. Input device 22 is configured to receive input from the clinician and produces input signals. Input device 22 may also be configured to generate feedback to the clinician. The feedback can be visual, auditory, haptic, or the like.

The workstation 12 can further include computing devices and/or controllers such as a master processor circuit 22a in communication with the input device 22 for receiving the input signals and generating control signals for controlling the robotic surgical system 10, which can be transmitted to the instrument cart 14 via an interface cable 22b. In some cases, transmission can be wireless and interface cable 22b may not be present. The input device 22 can include right and left-hand controls (not shown) and/or foot pedals (not shown), which are moved/operated to produce input signals at the input device 22 and/or to control robotic surgical system 10. The instrument cart 14 can include a slave processor circuit 20a that receives the control signals from the master processor circuit 22a and produces slave control signals operable to control the surgical instrument system 50 during a surgical procedure. The workstation 12 can also include a user interface, such as a display (not shown) in communication with the master processor circuit 22a for displaying information (such as, body cavity images) for a region or site of interest (for example, a surgical site, a body cavity, or the like) and other information to a clinician. While both master and slave processor circuits are illustrated, in other aspects, a single processor circuit may be used to perform both master and slave functions.

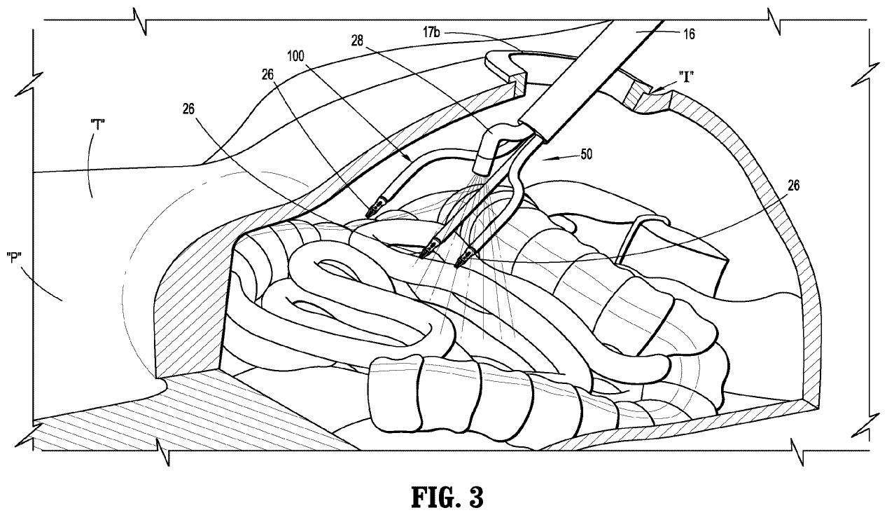

Surgical instrument system 50 of robotic surgical system 10 can include a plurality of surgical instruments 100. Each surgical instrument 100 is selectively attachable and selectively removable from moveable drive unit 18 (FIG. 1). Although only four surgical instruments 100 are shown in FIG. 3, for example, surgical instrument system 50 can include any number and/or type of surgical instruments.

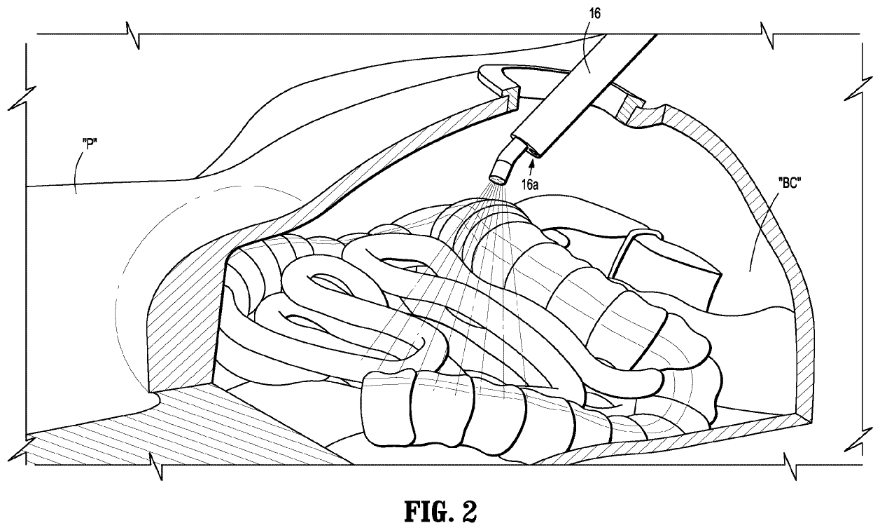

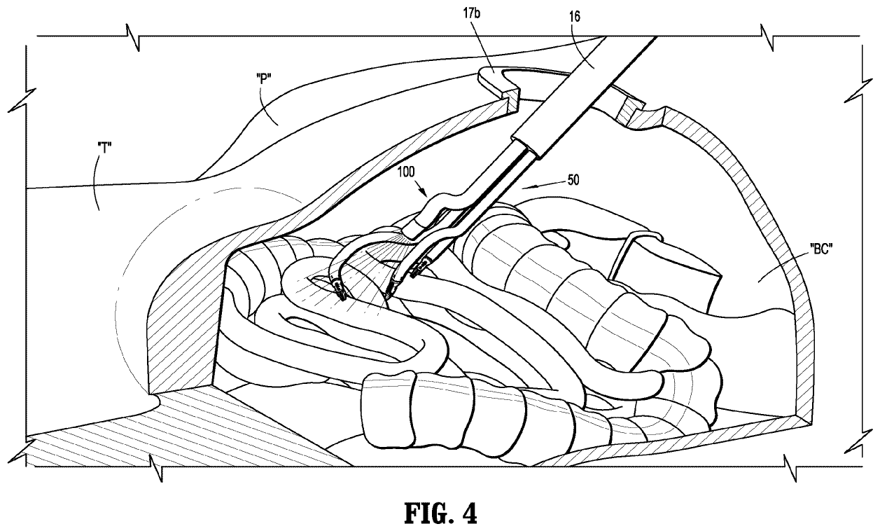

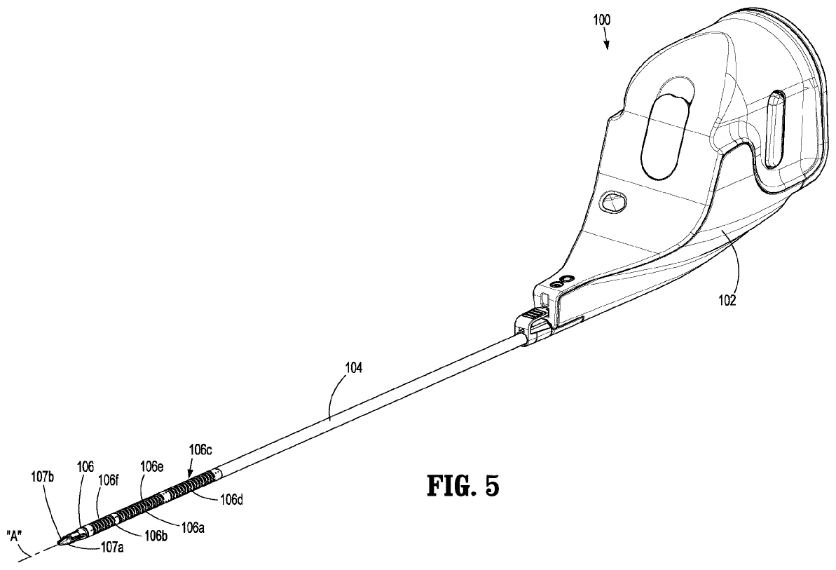

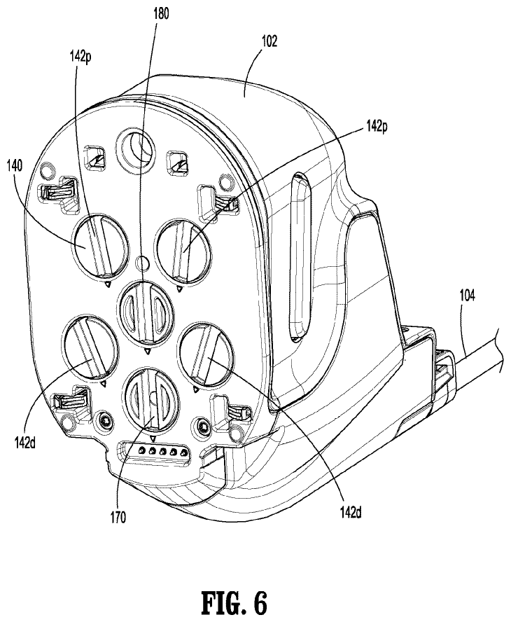

With reference to FIGS. 5 and 6, each surgical instrument 100 of surgical instrument system 50 defines a longitudinal axis “A” and includes an instrument cassette assembly 102 on a proximal end portion thereof, an elongated shaft assembly 104 that extends distally from instrument cassette assembly 102, and an end effector 106 supported on a distal end portion of elongated shaft assembly 104. End effector 106 may include portions (e.g., distal portions) of elongated shaft assembly 104, such as a dexterous assembly 106a, that connects elongated shaft assembly 104 to jaw members 107a, 107b of end effector 106 via a wrist 106b and facilitates movement of end effector 106 (e.g., upon a manipulation of one or more cables of a drive assembly of the surgical instrument 100). End effector 106 is actuatable by instrument cassette assembly 102 for effectuating a surgical procedure. Indeed, actuating end effector 106 can cause end effector 106 to, for example, articulate, pivot, clamp, rotate, etc. relative to the longitudinal axis “A” of surgical instrument 100 for repositioning end effector 106 and/or for treating tissue “T” of the patient “P” as noted above (see FIGS. 2-4). Dexterous assembly 106 includes a vertebral stack 106c that creates an alternating set of orthogonal joints such that every other joint allows bending in an XZ plane and every other joint allows bending in a YZ plane. Vertebral stack 106c includes sections 106d, 106e, and 106f.

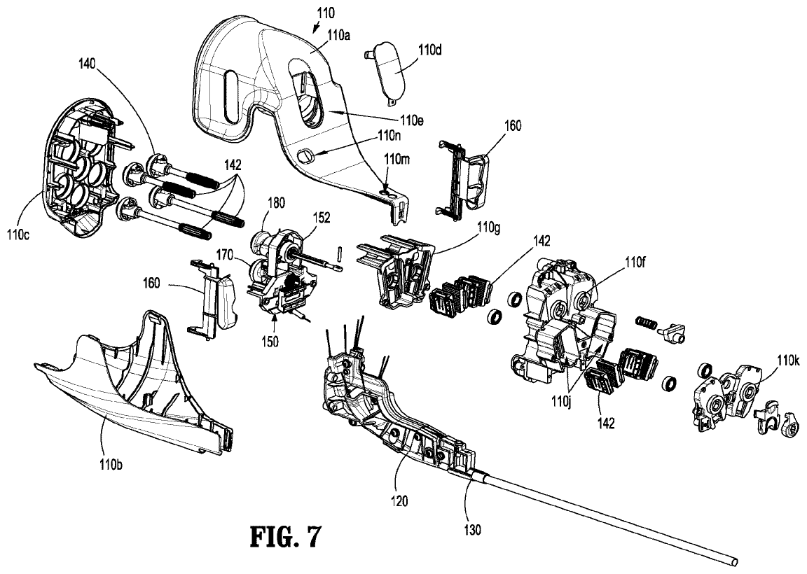

As seen in FIG. 7, instrument cassette assembly 102 includes a housing assembly 110, a transition block assembly 120, a shaft hub assembly 130, a cable drive assembly 140, a grip and wrist hub assembly 150, and a latch assembly 160.

Housing assembly 110 of instrument cassette assembly 102 includes an upper housing portion 110a, a lower housing portion 110b, and a rear housing portion 110c that couple together to support various components within housing assembly 110. Housing assembly 110 further includes a cover 110d that is removably attachable to a manual release opening 110e defined in upper housing portion 110a. Housing assembly 110 further includes a first support body 110f and a second support body 110g that are configured to support various internal components of instrument cassette assembly 102. Housing assembly 110 further includes a flush port 110m and a socket for an electrosurgical cable 110n.

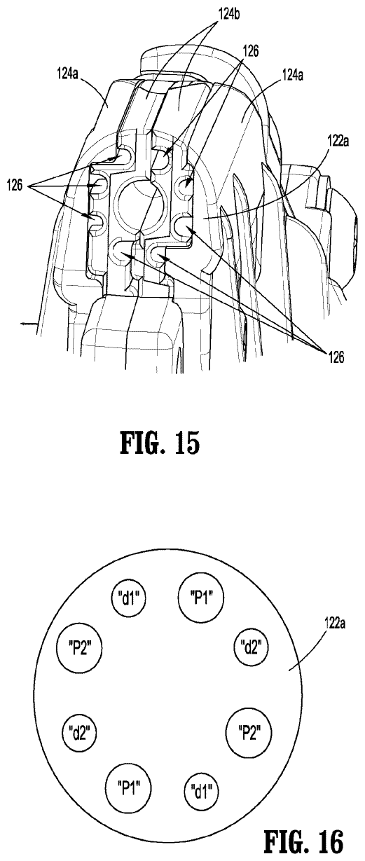

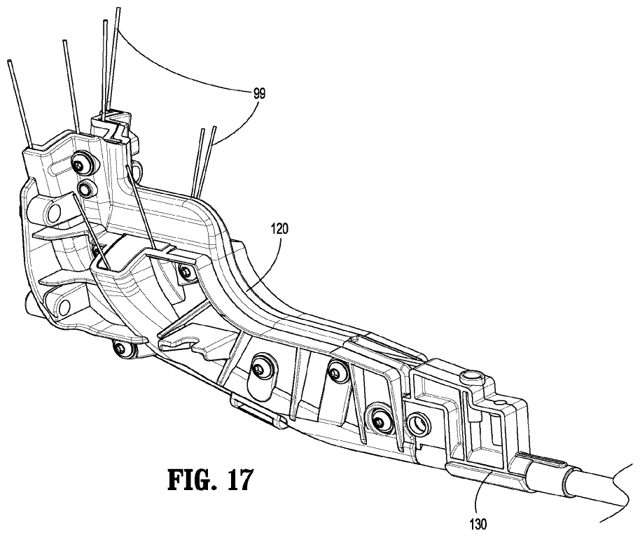

With reference to FIGS. 14, 15, 16, 17, and 18, transition block assembly 120 of instrument cassette assembly 102 includes a transition block 122 and supports a plurality of cables 99 therethrough. Transition block 122 of transition block assembly 120 includes outer shells 124a and inner shells 124b that couple together via fasteners (not explicitly shown) to define cable channels 126 therethrough for routing cables 99 in a longitudinal radial pattern through shaft hub assembly 130 on a first end 122a of transition block 122 and into housing assembly 110 toward cable drive assembly 140 in a radial fan pattern on a second end 122b of transition block 122 (see FIGS. 14-16). Indeed, cable pairs are routed through cable channels 126 in diametrically opposed relation (e.g., 180 degrees apart). For instance, as seen in FIG. 16, cable pairs “d1” cooperate with one another, cable pairs “P1” cooperate with one another, cable pairs “d2” cooperate with one another, and cable pairs “P2” cooperate with one another. Each of these cable pairs shown in FIG. 16 corresponds to the matching locations of cables channels 126 shown in FIG. 15. Outer and inner shells 124a, 124b may include or be formed of low coefficient of friction polyphenylene sulfide, for instance, to minimize drivetrain losses. Transition block 122 may have one or more bends therein to facilitate cable routing therethrough and to help reduce load distribution (e.g., frictional forces acting on such cables as cables translate therealong).

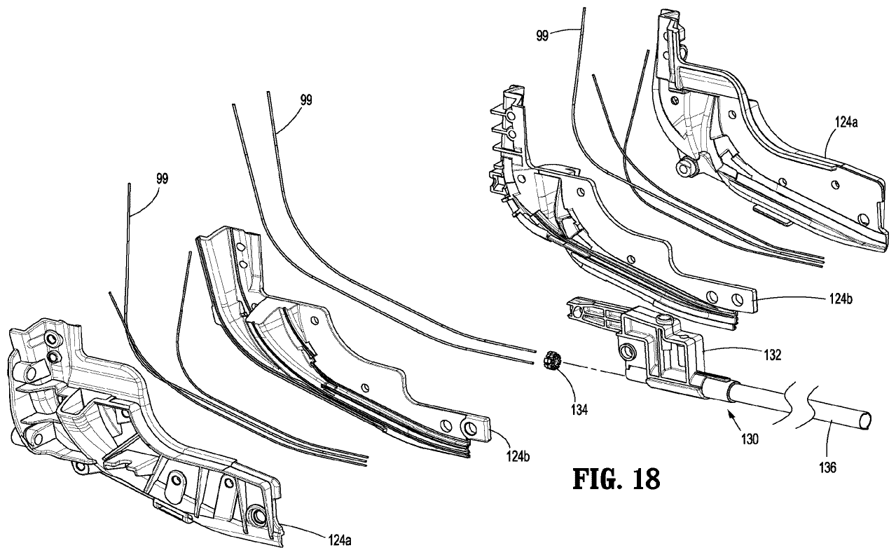

With continued reference to FIG. 18, shaft hub assembly 130 of instrument cassette assembly 102 includes a shaft hub 132 (e.g., polyetheretherketone) that supports a cable seal 134 (e.g., silicone) in a proximal portion thereof for sealing and guiding cables 99 through shaft hub 132. Shaft hub assembly 130 further includes a distal shaft 136 (e.g., stainless steel) that extends distally from shaft hub 132 and receives cables 99 therethrough.

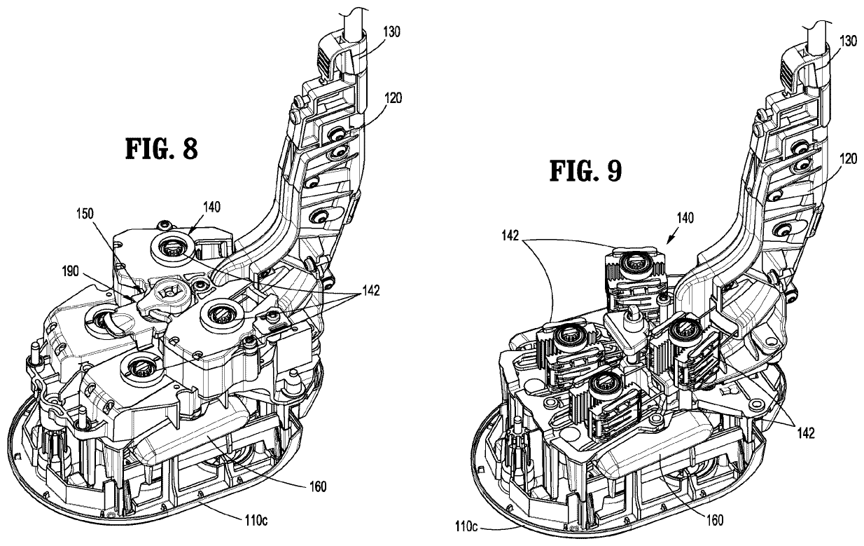

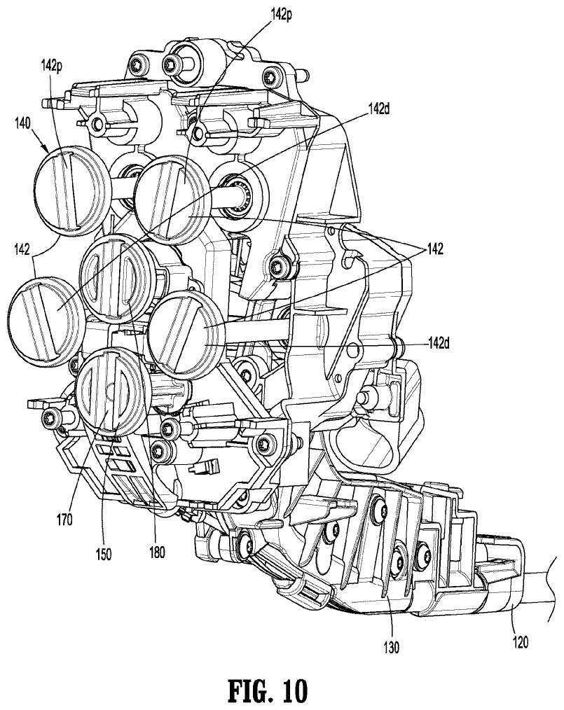

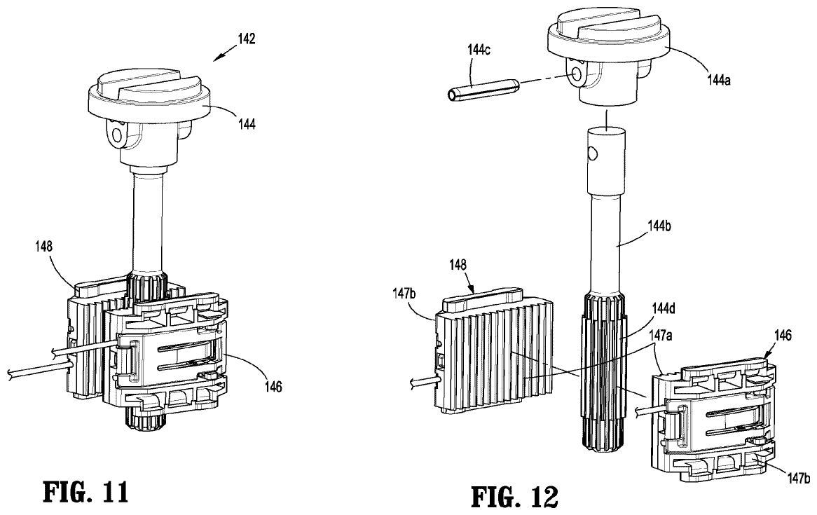

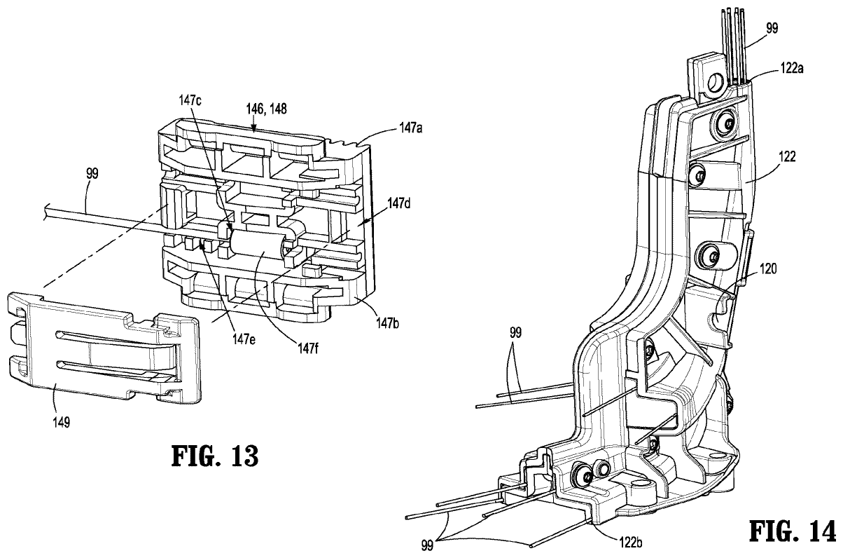

Turning now to FIGS. 6-13, cable drive assembly 140 of instrument cassette assembly 102 includes a plurality of drive dog assemblies 142. The plurality of drive dog assemblies 142 includes proximal drive dog assemblies 142p and distal drive dog assemblies 142d configured to impart movement of cables 99. As seen in FIGS. 11-13, each drive dog assembly 142 includes a drive dog pinion 144, a first rack assembly 146, and a second rack assembly 148 that are movably coupled together and positioned to impart movement to cables 99 coupled thereto. Drive dog pinon 144 includes a drive dog 144a (made e.g., from polyetheretherketone) and a pinion 144b (made e.g., from stainless steel) extending distally from drive dog 144a. Drive dog 144a is pinned to a proximal portion of pinion 144b by a pin 144c. Pinion 144b includes a plurality of teeth 144d about a circumference of a distal portion of pinion 144b. Each of the first and second rack assemblies 146, 148 (made e.g., from polyetheretherketone) includes a rack 147a on a first side thereof and a retaining portion 147b on a second side thereof that is opposite to the first side. Retaining portion 147b defines a ferrule opening 147c, a clip opening 147d, and a cable channel 147e therein. Ferrule opening 147c supports a ferrule 147f that retains an end of a cable 99 therein (e.g., cable 99 crimped by ferrule 147f with, for example, about 0.8 mm of preload applied). Cable channel 147e is in communication with ferrule opening 147c and supports cable 99 therein. A clip 149 is selectively received within clip opening 147d and removably secured to retaining portion 147b (e.g., snap-fit) to secure ferrule 147f and cable 99 within cable channel 147e and ferrule opening 147c, respectively.

As best illustrated in FIG. 7, the first and second rack assemblies 146, 148 of proximal drive dog assemblies 142p are supported within rack cavities 110j defined within first support body 110f of housing assembly 110. Rack cavities 110j are enclosed with a lid assembly 110k. The first and second rack assemblies 146, 148 of distal drive dog assemblies 142d are supported within and disposed between first and second support bodies 110f, 110g.

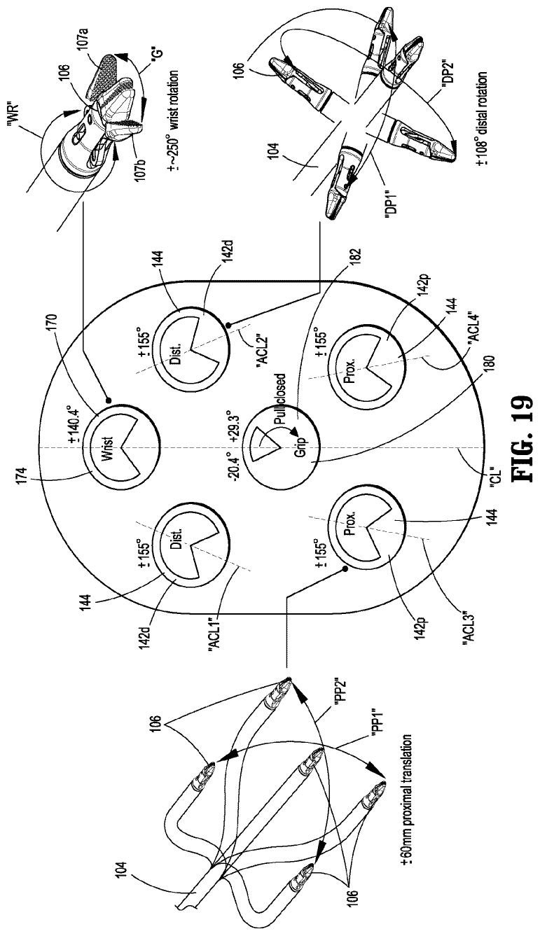

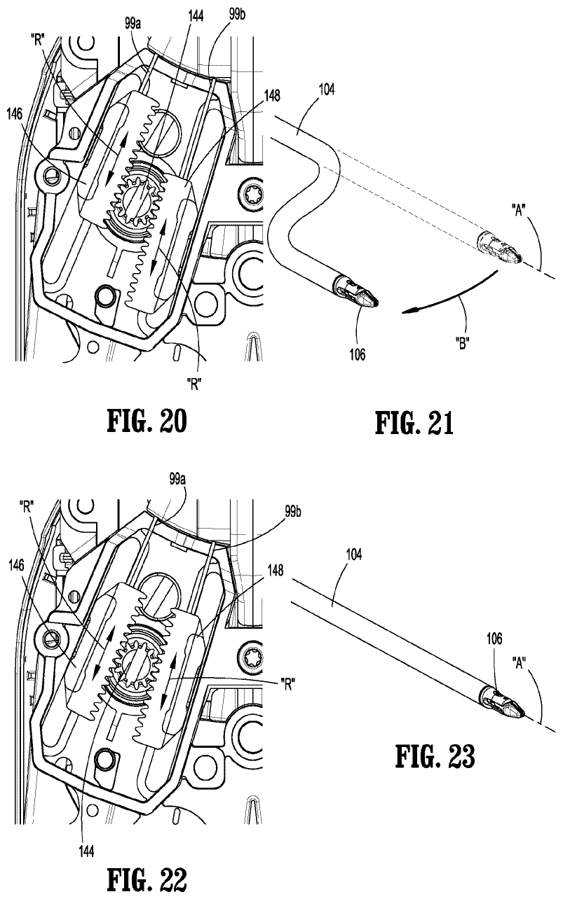

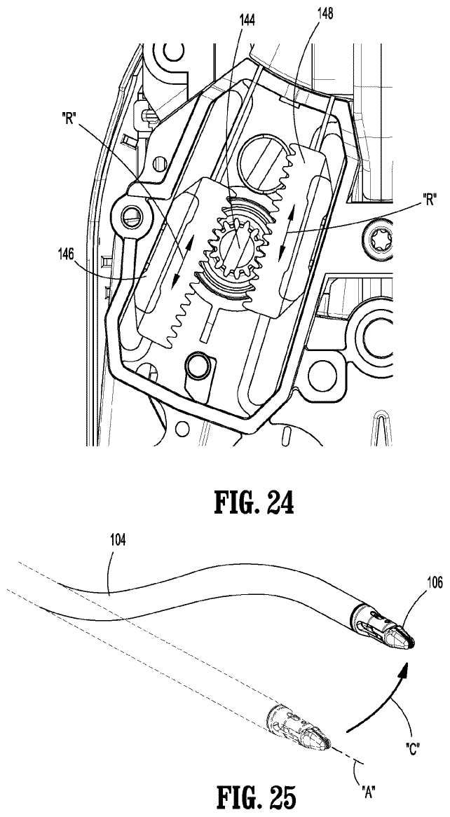

As seen in FIGS. 20-25, rotation of each drive dog pinion 144 causes first and second racks 146, 148 to move (e.g., translate relative to one another, for instance, up to 9 mm of travel before hitting a hard stop), as indicated by arrows “R”, to cause first and second cables 99a, 99b coupled to racks 146, 148, respectively, to translate relative to one another to generate a push-pull force for imparting motion/force to the end effector 106. Relative movement of racks 146, 148 and first and second cables 99a, 99b causes end effector 106 to move relative to longitudinal axis “A,” as indicated by arrows “B” and “C” shown in FIGS. 21 and 25. Notably, when racks 146, 148 are substantially aligned with one another (e.g., disposed in mirrored relation to one another), end effector 106 is disposed in alignment with longitudinal axis “A” as seen in FIG. 23. As seen in FIG. 19, drive dog pinons 144 of proximal and distal drive dog assemblies 142p, 142d are configured to rotate up to about 155 degrees in clockwise and/or counterclockwise directions relative to respective angled centerlines “ACL1” to “ACL4” for moving end effector 106 through and/or relative to proximal and/or distal planes “PP1” (e.g., rotation of a first proximal drive dog assembly) “PP2,” (e.g., rotation of a second proximal drive dog assembly) “DP1,” (e.g., rotation of a first distal drive dog assembly) and “DP2” (e.g., rotation of a second distal drive dog assembly) shown in FIG. 19. In particular, translational movement of end effector 106 relative to/through proximal planes “PP1,” “PP2,” may be up to 60 mm in proximal and/or distal directions, and rotational movement of end effector 106 through/relative to distal planes “DP1” and “DP2” may be up to 108 degrees in clockwise and/or counterclockwise directions.

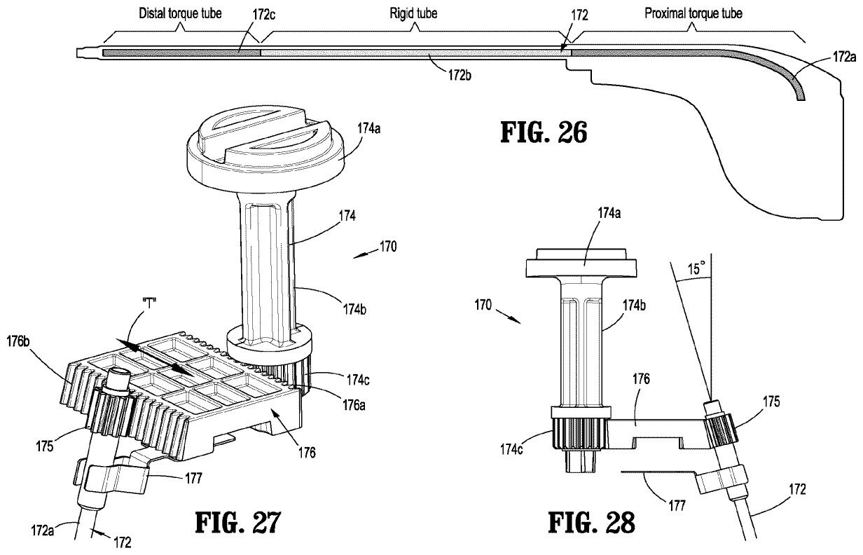

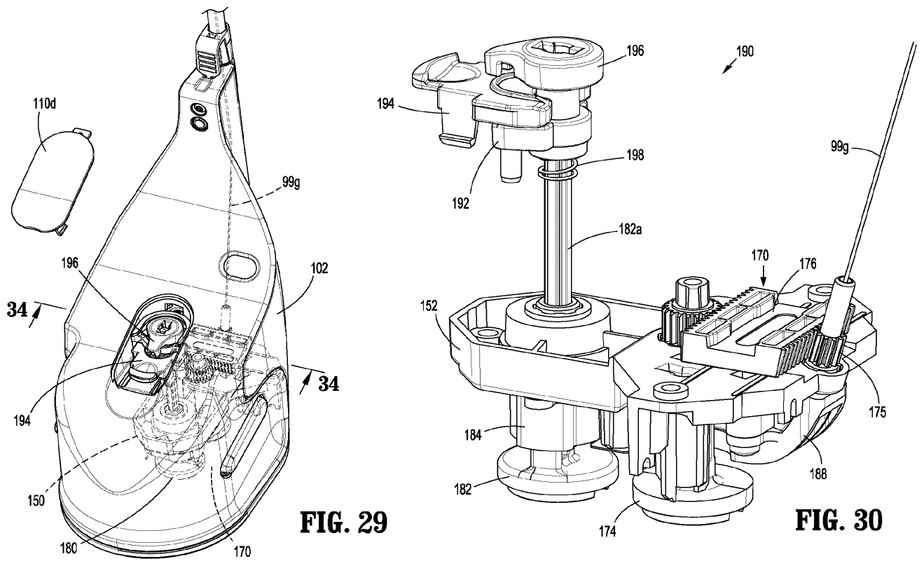

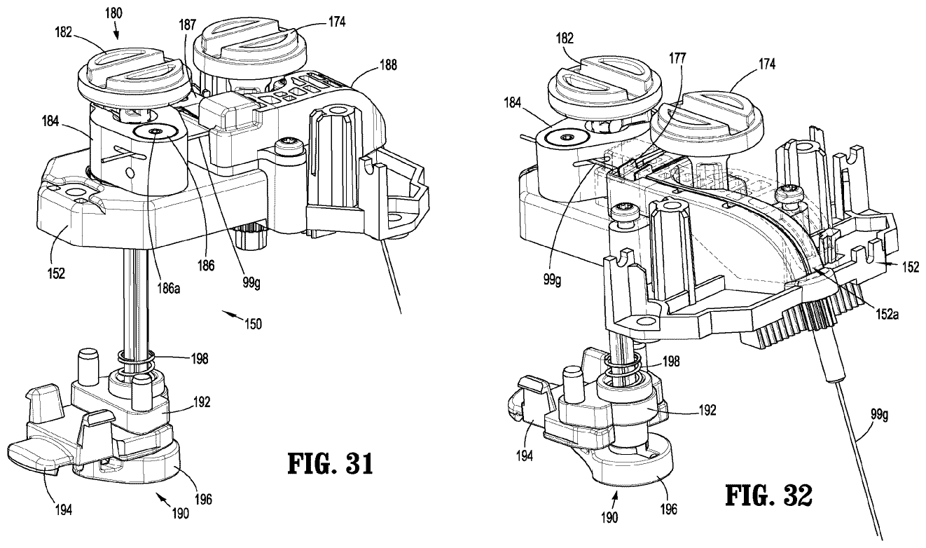

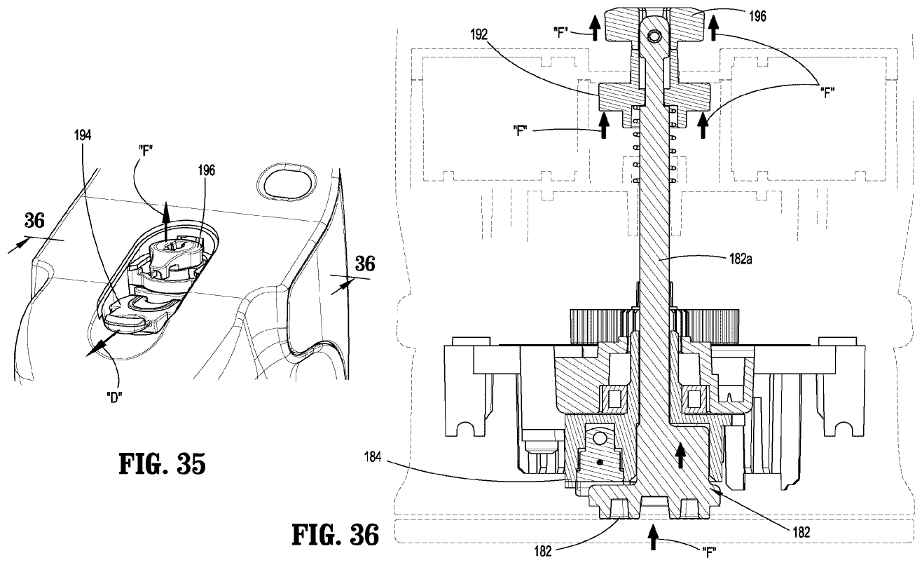

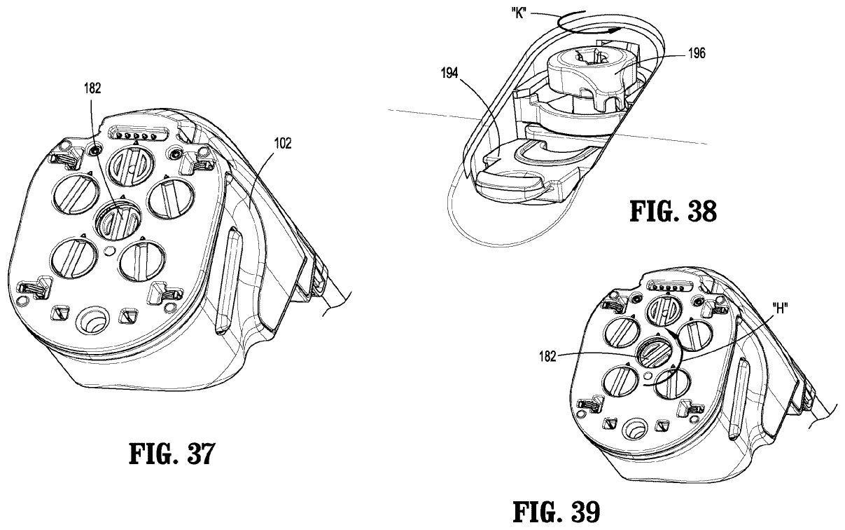

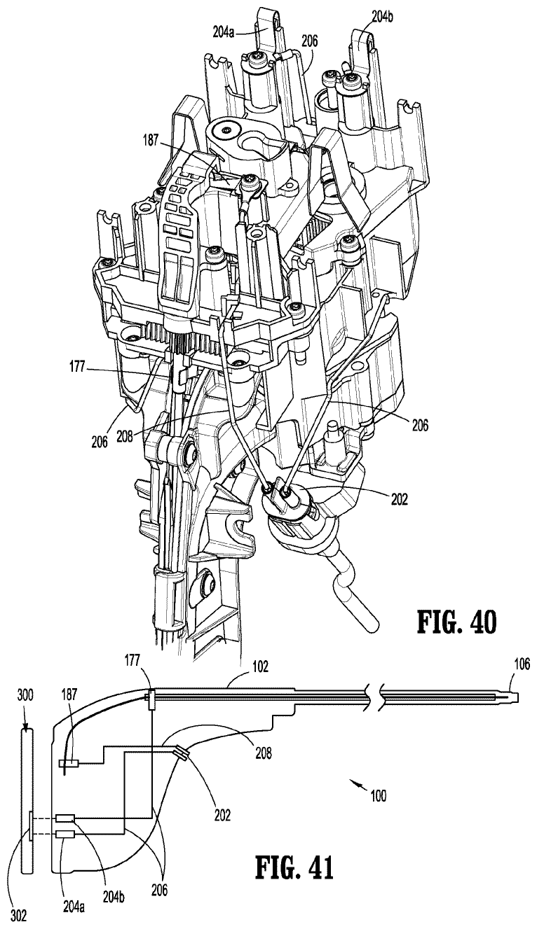

With reference to FIGS. 6, 7, 19, 26-39, grip and wrist hub assembly 150 includes a wrist mechanism 170 and a grip mechanism 180 that are supported by a mounting plate 152 within housing assembly 110. Grip and wrist hub assembly 150 further includes a manual release mechanism 190 coupled to grip mechanism 180 and a linear wiper 187 of a high voltage circuit supported on mounting plate 152.

Wrist mechanism 170 of grip and wrist hub assembly 150 provides a wrist roll function that is achieved by a compounded shaft assembly 172 that extends through elongated shaft assembly 104. Compound shaft assembly 172 includes a proximal flexible section 172a, a rigid intermediate section 172b, and a distal flexible section 172c that are secured together by, for instance, laser welding. Compounded shaft assembly 172 may be tubular. Distal flexible section 172c is disposed in dexterous portion 106a of end effector 106 for transferring rotation through dexterous portion 106a. Distal flexible section 172c may include a tri-layer stainless steel weave component that flexes radially while efficiently transferring torque axially to the wrist 106b of end effector 106.

Wrist mechanism 170 further includes a drive dog pinion 174 (made e.g., of a polyphenylene sulfide such as Fortron®) having a drive dog 174a on first end thereof and a shaft 174b extending distally from drive dog 174a. Shaft 174b supports a pinion 174c on a distal end portion of shaft 174b. Pinion 174c functions as an input gear. Wrist mechanism 170 further includes a double-sided rack 176 (made e.g., from polyetheretherketone) having a first side rack 176a on a first side of double-sided rack 176 and a second side rack 176b on a second side of double-sided rack 176. Wrist mechanism 170 further includes a wrist gear 175 (made e.g., of stainless steel) coupled to a first end of proximal flexible section 172a of compounded shaft assembly 172. Wrist gear 175, which functions as an output gear, is enmeshed with second side rack 176b, and pinion 174c of drive dog pinion 174 is enmeshed with first side rack 176a, wherein rotation of drive dog pinion 174 rotates pinion 174c and causes double-sided rack 176 to translate relative to drive dog pinion 174 and wrist gear 175, as indicated by arrows “T.” As double-sided rack 176 translates, double-sided rack 176 rotates wrist gear 175, via a 2:1 gearing ratio, for example, causing compounded shaft assembly 172 to rotate and impart a wrist-roll movement to end effector 106. First side rack 176a is parallel to pinion 174c and second side rack 176b is disposed at an angle (e.g., 15 degrees, although any suitable angle may be provided) relative to first side rack 176a. Wrist gear 175 further supports a rotary contact wiper 177 of the high voltage circuit, and which may include gold-plated, stainless steel, high-voltage material. As seen in FIG. 19, drive dog pinon 174 may be rotatable up to about 140.4 degrees in clockwise and/or counterclockwise directions relative to a centerline “CL” of instrument cassette assembly 102 to effectuate wrist rotation “WR” of end effector 106 up to about 250 degrees in clockwise and/or counterclockwise directions.

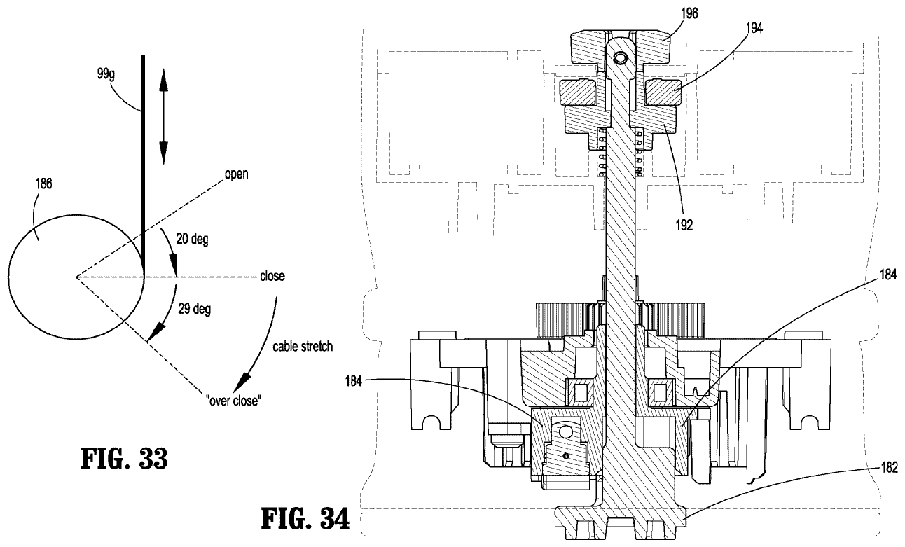

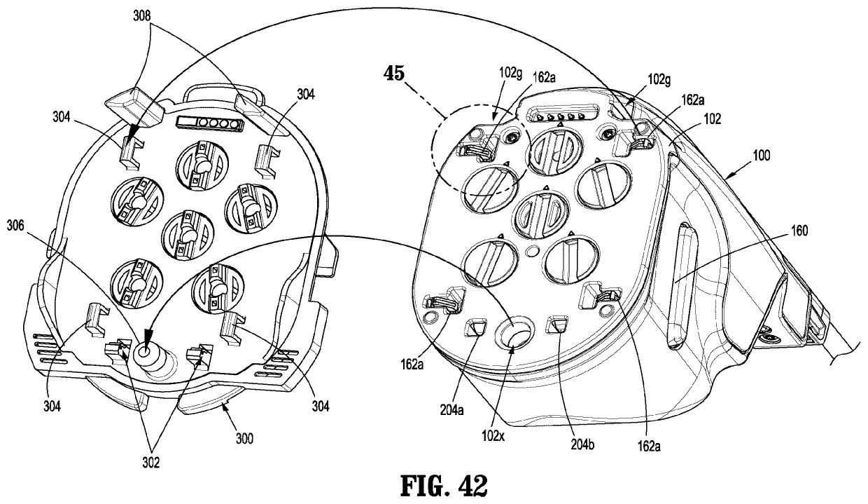

Grip mechanism 180 of grip and wrist hub assembly 150 includes a drive dog 182 and a crank 184 supported about drive dog 182, both of which may include or be formed of polyetheretherketone. Drive dog 182 is configured to receive motor input and engage/disengage with a sterile adapter assembly 300 (FIG. 42) during manual grip release. Drive dog 182 includes a drive dog shaft 182a that supports manual release mechanism 190 thereon. Grip mechanism 180 further includes a grip cable 99g that extends through crank 184. Grip cable 99g is secured to crank 184 by a pin 186 (e.g., stainless steel) and set screw 186a such that rotation of drive dog 182 causes crank 184 to rotate and impart movement on grip cable 99g to effectuate the gripping movement “G” of jaw members 107a, 107b. Grip mechanism 180 further includes a wire routing cap 188 that is secured to mounting plate 152 and constrains grip cable 99g and supports grip cable 99g within a channel 152a defined in mounting plate 152. With reference to FIGS. 19 and 33, grip mechanism 180 provides significant additional travel due to cable stretch when applying high grip loads. Indeed, drive dog 182 is rotatable, relative to centerline “CL” of instrument cassette assembly 102, up to 20.4 degrees in a counterclockwise direction (e.g., opening) and up to 29.3 degrees in a clockwise direction (e.g., a pull closed direction) to effectuate gripping movement “G” of jaw members 107a, 107b. Jaw members 107a, 107b are closed when drive dog 182 is disposed at zero degrees, and when drive dog 182 is rotated clockwise beyond the zero degree position (up to the 29.3 degrees), jaw members 107a, 107b are disposed in overclosed positions. In such overclosed positions, grip cable 99g stretches.

Manual release mechanism 190 of instrument cassette assembly 102 includes a carriage 192, a lock sled 194, a dial 196, and a spring 198. Lock sled 194 is slidably coupled to carriage 192 to enable manual release mechanism 190 to move from a first or normal state (FIG. 34) to a second or release state (FIG. 36). To effectuate manual release, cover 110d is removed from instrument cassette assembly 102 to access manual release mechanism 190. Lock sled 194 is then pulled out from manual release mechanism 190, as indicated by arrow “D,” so that dial 196, drive dog 182, and carriage 192 are urged upward by spring 198, as indicated by arrows “F,” causing drive dog 182 to recess within instrument cassette assembly 102 and disengage from a motor of instrument drive assembly 20 to prevent gearbox back-drive and low torque actuation of manual release. Dial 196 is then rotated relative to carriage 192, as indicated by arrow “K” (e.g., approximately 20 degrees), to open grip of jaw members 107a, 107b of end effector 106, whereby drive dog 182 concomitantly rotates, as indicated by arrow “H.”

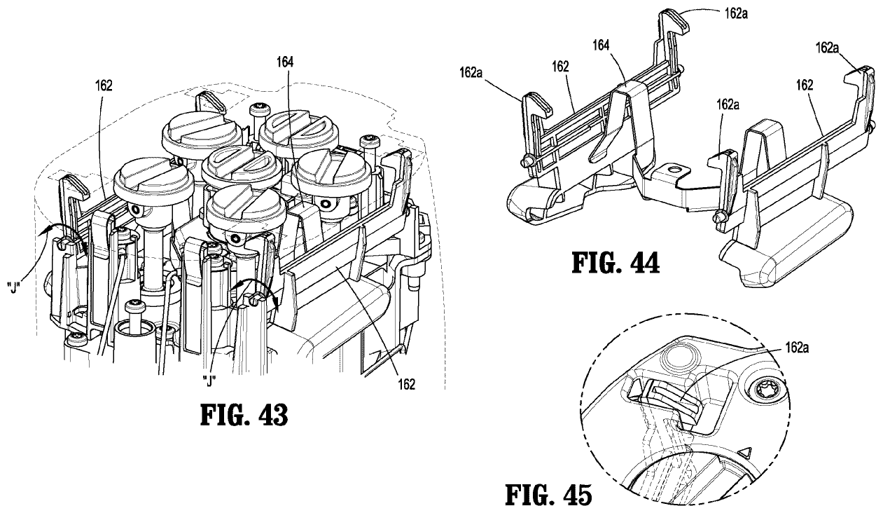

With reference to FIGS. 41-45, instrument cassette assembly 102 of robotic surgical instrument 100 further includes a high voltage circuit 200 including a bipolar connector 202, grip wiper 187, wrist wiper 177, and sterile adapter bridge contacts 204a, 204b that are coupled together via wires 206 and 208. High voltage circuit 200 is configured to deliver high voltage to end effector 106, for instance, to effectuate tissue sealing. Sterile adapter bridge contacts 204a, 204b are configured to engage with a sterile adapter bridge 302 supported on sterile adapter assembly 300 to enable electrical energy to transfer from, for example, instrument drive assembly 20 of movable drive unit 18, through sterile adapter assembly 300, and to surgical instrument 100.

Sterile adapter assembly 300 of robotic surgical system 10 is configured to couple to a proximal end portion of instrument cassette assembly 102 via a latch assembly 160. Latch assembly 160 includes latches 162 and a latch spring 164 that couples latches 162 together. Latches 162 include hooks 162a that are pivotably mounted in instrument cassette assembly 102 and positioned to releasably lock instrument cassette assembly 102 onto sterile adapter assembly 300 via latch clips 304 of sterile adapter assembly 300. Actuation of latch assembly 1600 causes latches 162 to pivot, as indicated by arrows “J,” thereby releasing instrument cassette assembly 102 from sterile adapter assembly 300. Sterile adapter assembly 300 further includes an X-Y location pin 306 that is received in a location pin aperture 102x defined in instrument cassette assembly 102, and catch ribs 308 that engage rotation constraint channels 102y defined by instrument cassette assembly 102 to facilitate an interface between sterile adapter assembly 300 and surgical instrument 100

The phrases “in an aspect,” “in aspects,” “in various aspects,” “in some aspects,” or “in other aspects” may each refer to one or more of the same or different aspects in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

Securement of any of the components of the disclosed devices may be effectuated using known securement techniques such welding, crimping, gluing, fastening, etc.

Persons skilled in the art will understand that the structures and methods specifically described herein and shown in the accompanying figures are non-limiting exemplary aspects, and that the description, disclosure, and figures should be construed merely as exemplary of aspects. It is to be understood, therefore, that this disclosure is not limited to the precise aspects described, and that various other changes and modifications may be effectuated by one skilled in the art without departing from the scope or spirit of the disclosure. Additionally, the elements and features shown or described in connection with certain aspects may be combined with the elements and features of certain other aspects without departing from the scope of this disclosure, and that such modifications and variations are also included within the scope of this disclosure. Accordingly, the subject matter of this disclosure is not limited by what has been particularly shown and described.

What is claimed is:

1. A surgical instrument for a robotic surgical system, the surgical instrument comprising:
an end effector;
an elongated shaft assembly defining a longitudinal axis and having a distal end portion and a proximal end portion, the distal end portion including a dexterous assembly supporting the end effector, the dexterous assembly being movable relative to the longitudinal axis to move the end effector in different planes; and
an instrument cassette assembly supported on the proximal end portion of the elongated shaft assembly, the instrument cassette assembly including a cable drive assembly including a plurality of drive dog assemblies, each drive dog assembly of the plurality of drive dog assemblies including a drive dog pinion, a first rack assembly, and a second rack assembly, the first rack assembly including a first cable, the second rack assembly including a second cable, the first and second rack assemblies positioned to translate relative to one another in response to rotation of the drive dog pinion to cause the first and second cables to move the end effector relative to the longitudinal axis.

2. The surgical instrument of claim 1, wherein the first rack assembly includes a first ferrule that secures the first cable to a first rack of the first rack assembly.

3. The surgical instrument of claim 2, wherein the first rack assembly includes a clip that is removably coupled to the first rack to secure the first ferrule to the first rack.

4. The surgical instrument of claim 1, wherein the instrument cassette assembly includes a transition block that directs the first and second cables into the elongated shaft assembly.

5. The surgical instrument of claim 4, wherein the transition block includes a plurality of shells secured together to define cable channels through the transition block, wherein the cable channels define a longitudinal radial pattern that is configured to direct the first and second cables into a shaft hub assembly supported on the proximal end portion of the elongated shaft assembly.

6. The surgical instrument of claim 1, wherein the instrument cassette assembly includes a wrist mechanism, the wrist mechanism including a double-sided rack, a wrist drive dog pinion, and a wrist gear, the wrist drive dog pinon being rotatable to translate the double-sided rack, the wrist gear being rotatable in response to translation of the double-sided rack.

7. The surgical instrument of claim 6, wherein the wrist mechanism further includes a compound shaft assembly coupled to the wrist gear.

8. The surgical instrument of claim 7, wherein the compound shaft assembly extends through the elongated shaft assembly and includes at least one flexible section and at least one rigid section.

9. The surgical instrument of claim 6, wherein the wrist gear is disposed at an angle relative to the wrist drive dog pinon.

10. The surgical instrument of claim 1, wherein the instrument cassette assembly includes a grip mechanism that is actuatable to move jaw members of the end effector relative to another, and a manual release mechanism that is coupled to the grip mechanism.

11. An instrument cassette assembly of a robotic surgical instrument, the instrument cassette assembly comprising:
a housing assembly supporting a mounting plate;
a cable drive assembly supported in the housing assembly and including a drive dog pinion, a first rack assembly, and a second rack assembly, the first and second rack assemblies enmeshed with the drive dog pinion and positioned to translate relative to one another in response to rotation of the drive dog pinion;
a grip mechanism supported on the mounting plate; and
a release mechanism coupled to the grip mechanism.

12. The instrument cassette assembly of claim 11, wherein the housing assembly includes a cover that is selectively removable from the housing assembly to expose the release mechanism.

13. The instrument cassette assembly of claim 12, wherein the release mechanism includes a lock sled, a carriage, a dial, and a spring, the lock sled slidably movable relative to the carriage to enable the spring to urge the carriage and the dial from a first position to a second position.

14. The instrument cassette assembly of claim 13, wherein the dial is rotatable relative to the carriage in the second position to operate the grip mechanism.

15. The instrument cassette assembly of claim 11, wherein the grip mechanism includes a drive dog and a crank operatively associated with the drive dog, the crank configured to receive a grip cable therethrough.

16. The instrument cassette assembly of claim 11, further comprising a high voltage circuit supported within the housing assembly.

17. The instrument cassette assembly of claim 11, further comprising a latch assembly supported by the housing assembly, the latch assembly being actuatable to disconnect a sterile adapter assembly from the housing assembly.

18. The instrument cassette assembly of claim 17, wherein the latch assembly includes a latch spring coupled to a pair of latches, the pair of latches configured to secure the housing assembly to the sterile adapter assembly.

19. The instrument cassette assembly of claim 18, further comprising the sterile adapter assembly.

20. A robotic surgical system, comprising:
an instrument drive assembly;
a sterile adapter assembly coupled to the instrument drive assembly; and
a robotic surgical instrument coupled to the sterile adapter assembly and actuatable by the instrument drive assembly, the robotic surgical instrument including an instrument cassette assembly, an elongated shaft assembly extending from the instrument cassette assembly, and an end effector supported on a distal end portion of the elongated shaft assembly, the elongated shaft assembly defining a longitudinal axis, the instrument cassette assembly including:
a cable drive assembly including a plurality of drive dog assemblies, each drive dog assembly of the plurality of drive dog assemblies including a drive dog pinion, a first rack assembly, and a second rack assembly, the first and second rack assemblies positioned to translate relative to one another in response to rotation of the drive dog pinion to cause the end effector to move relative to the longitudinal axis;
a grip mechanism operatively coupled to the end effector and actuatable to cause jaw members of the end effector to move between an open position, a closed position, and an overclosed position;
a manual release mechanism operatively coupled to the grip mechanism to enable the jaw members to be manually moved relative to one another; and
a wrist mechanism operatively coupled to the end effector and actuatable to cause the end effector to rotate.
